(19) United States
(12) Patent Application Publication
(10) Pub. No.: US 2018/0330804 A1
(43) Pub. Date: Nov. 15, 2018

(54) PASSENGER HEALTH MANAGEMENT APPARATUS, PASSENGER HEALTH MANAGEMENT SYSTEM AND PASSENGER HEALTH MANAGEMENT METHOD

(71) Applicant: Honda Motor Co., Ltd., Tokyo (JP)

(72) Inventors: Shigenori Hiruta, Wako-shi (JP); Shiro Kitamura, Wako-shi (JP); Kimiyoshi Kusaka, Wako-shi (JP)

(21) Appl. No.: 15/970,804

(22) Filed: May 3, 2018

(30) Foreign Application Priority Data
May 10, 2017 (JP) 2017-094126

(51) Int. Cl. G16H 10/60; G16H 50/30; B60W 40/08; A61B 5/00

(52) U.S. Cl. CPC G16H 10/60; G16H 50/30; A61B 5/02055; A61B 5/6893; B60W 40/08

(57) ABSTRACT

A passenger health management apparatus, including: a measuring unit configured to measure health condition of a passenger onboard to acquire a biometric data; a CPU and a memory coupled to the CPU; and a presentation unit configured to preform presentation to the passenger, the CPU and the memory being configured to determine a proposal to be presented to the passenger from among a plurality of proposals regarding passenger health management including a proposal advising medical examination at a medical facility and a proposal advising purchase of medicine at a shop that sells medicine based on the biometric data acquired by the measuring unit, wherein the presentation unit is further configured to present the proposal to the passenger.

### CROSS-REFERENCE TO RELATED APPLICATION

This application is based upon and claims the benefit of priority from Japanese Patent Application No. 2017-094126 filed on May 10, 2017, the content of which is incorporated herein by reference.

### BACKGROUND OF THE INVENTION

### Field of the Invention

This invention relates to a passenger health management apparatus, passenger health management system and passenger health management method which measures health condition of a passenger in a vehicle and manages the passenger's health.

### Description of the Related Art

Apparatuses are known that are adapted to evaluate health condition of a passenger or passengers in a vehicle, determine based on the result of the evaluation whether any passenger should be examined at a medical facility, and apply for a reservation at the passenger's primary care hospital or at a nearby hospital when examination at a medical facility is determined to be advisable (see Japanese Unexamined Patent Publication No. 2006-302206 (JP2006-302206A),

However, since the apparatus taught by JP2006-302206A does nothing beyond determining whether examination at a medical facility is advisable, it is not capable of offering a proper proposal to a passenger when, for example, the symptoms of the illness are mild and not so severe as to require examination at a medical facility.

### SUMMARY OF THE INVENTION

An aspect of the present invention is a passenger health management apparatus, including: a measuring unit configured to measure health condition of a passenger onboard to acquire a biometric data; a CPU and a memory coupled to the CPU; and a presentation unit configured to preform presentation to the passenger, the CPU and the memory being configured to determine a proposal to be presented to the passenger from among a plurality of proposals regarding passenger health management including a proposal advising medical examination at a medical facility and a proposal advising purchase of medicine at a shop that sells medicine based on the biometric data acquired by the measuring unit, wherein the presentation unit is further configured to present the proposal to the passenger.

Another aspect of the present invention is a passenger health management system, including: an onboard unit having a measuring unit configured to measure health condition of a passenger onboard to acquire a biometric data; and a passenger health management server configured to be communicative with the onboard unit, wherein the passenger health management server has: a CPU and a memory coupled to the CPU, the CPU and the memory being configured to perform acquiring a biometric data acquired by the measuring unit, and determining a proposal to be presented to a passenger from among a plurality of proposals regarding passenger health management including a proposal advising medical examination at a medical facility and a proposal advising purchase of medicine at a shop that sells medicine based on the biometric data acquired by the measuring unit, wherein the onboard unit further has a presentation unit configured to acquire the proposal and to present the acquired proposal to the passenger.

Further aspect of the present invention is a passenger health management method, including: measuring health condition of a passenger onboard and acquiring a biometric data; determining a proposal to be presented to a passenger from among a plurality of proposals regarding passenger health management including a proposal advising medical examination at a medical facility and a proposal advising purchase of medicine at a shop that sells medicine based on the biometric data; and presenting the proposal to the passenger.

### BRIEF DESCRIPTION OF THE DRAWINGS

The objects, features, and advantages of the present invention will become clearer from the following description of embodiments in relation to the attached drawings, in which:

FIG. 1 is a diagram showing an example application of a passenger health management apparatus according to an embodiment of the present invention;

FIG. 2 is a block diagram showing a configuration overview of the passenger health management apparatus in accordance with the embodiment of the present invention;

FIG. 3 is a diagram showing examples of normal and abnormal ranges;

FIG. 4 is a flowchart showing an example of processing performed by an ECU of FIG. 2; and

FIG. 5 is a block diagram showing an example of the overall configuration of a passenger health management system in accordance with the embodiment of the present invention.

### DETAILED DESCRIPTION OF THE INVENTION

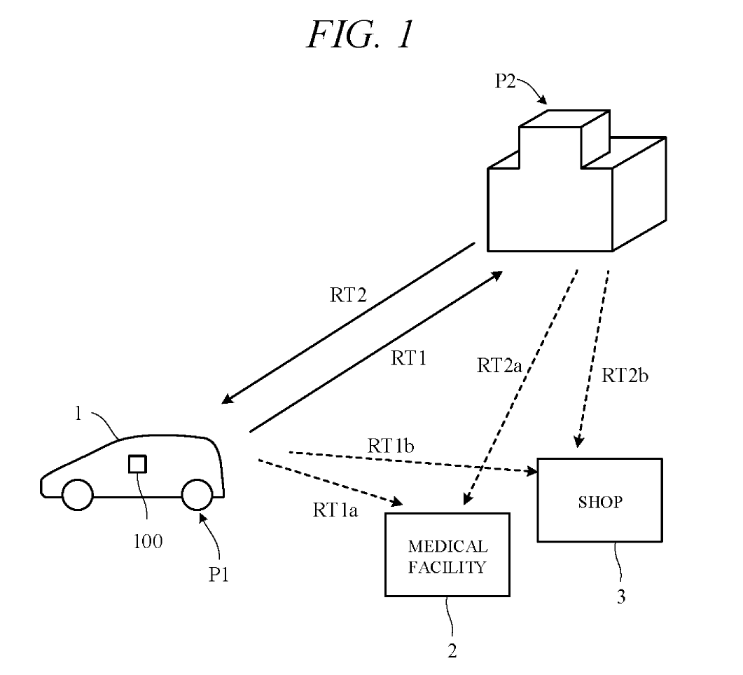

An embodiment of the present invention is explained below with reference to FIGS. 1 to 5. FIG. 1 is a diagram showing an example application of a passenger health management apparatus 100 according to the embodiment of the present invention. As seen in FIG. 1, the passenger health management apparatus 100 is, as one example, installed in a vehicle 1 having a navigation unit in which a target route from a departure point to a destination can be set.

Take, for example, a case in which the vehicle 1 drives along an outbound route RT1 from a departure point (e.g., residence) P1 to a destination (e.g., workplace or place to be visited) P2 and later along a return route RT2 from the destination P2 to the departure point P1. In such a case, the passenger health management apparatus 100 acquires passenger biometric data by measuring passenger health state factors such as body temperature and blood pressure inside the vehicle 1, and depending on the acquired biometric data, may, while on the outbound route RT1 or the return route RT2, advise a passenger to visit a hospital or medical facility 2 or to buy medicine at a drug store, chemist or other shop where pharmaceutical are sold.

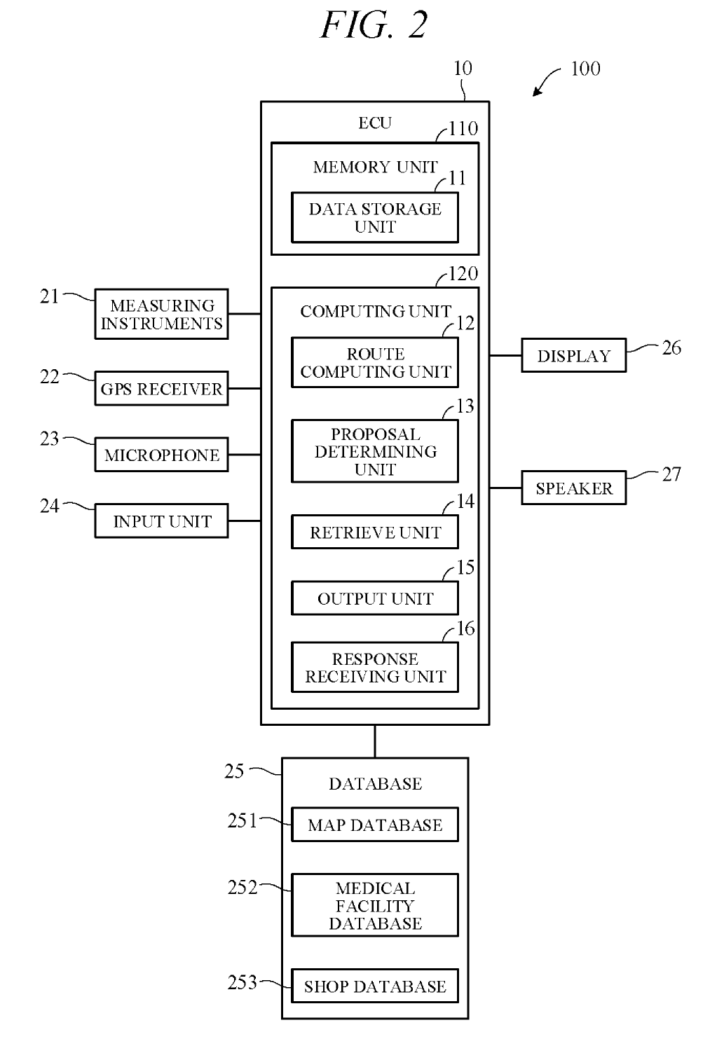

FIG. 2 is a block diagram showing a configuration overview of a passenger health management apparatus 100 in accordance with an embodiment of the present invention. The passenger health management apparatus 100 shares some functions with a navigation unit. As shown in FIG. 2, the passenger health management apparatus 100 comprises an ECU (electronic control unit) 10, and measuring instruments 21, a GPS receiver 22, a microphone 23, an input unit 24, a database 25, a display 26, and a speaker 27, which are wire or wirelessly electrically connected to the ECU 10.

The measuring instruments 21 comprise various contact and contactless sensors installed in the vehicle 1 for obtaining biometric data by measuring passenger health condition at preset times. The biometric data include data regarding passenger body temperature, blood pressure, pulse rate and other physical quantities related to passenger health condition. As specific examples of usable measuring instruments 21 can be cited, inter alia, one or more thermosensors for measuring passenger body temperature, electrodes attached to a steering wheel for measuring passenger (driver) heart rate and/or blood pressure, and cameras for measuring heart rate and/or pulse by passenger face imaging. The preset times for measuring biometric data can, for example, be when a passenger (in actual practice, driver) boards the vehicle or upon elapse of a predetermined time from start off. Alternatively, biometric data can be acquired at predetermined time intervals after passenger boarding.

The GPS receiver (GPS sensor) 22 can measure current vehicle position using signals it receives from GPS satellites. The microphone 23 acquires passenger voice utterances and produces corresponding voice signals that are converted to voice data by an A/D converter, for example, and input to the ECU 10 for voice recognition. The input unit 24 is a unit used by a passenger to input various instructions and can, for example, be configured as a touch panel or as a switch(es) provided on the steering wheel, for instance.

The database 25 includes a map database 251 for storing map data, a medical facility database 252 for storing medical facility data, and a shop database 253 for storing shop data. These data can be acquired from outside the vehicle 1 through a wireless or wire communication means and stored in the database 25. Data acquired from outside in this way can be updated at arbitrary or scheduled times.

Map data stored in the map database 251 include position, shape and other road data, as well as facility and shop location and footprint information and information about parking availability and so on. Medical facility data stored in the medical facility database 252 include data regarding location, medical departments, service hours, closed days, telephone numbers, parking lot availability, and other information on hospitals, clinics and other medical facilities. Shop data stored in the shop database 253 include data regarding location, in-shop pharmacist availability, business hours, closed days, telephone number, parking availability, and so on of drug stores, chemists, convenience stores and other shops where pharmaceuticals are sold. The shop information also includes information about types of medicines individual shops can handle, namely, which types are available among pharmacist-dispensed pharmaceuticals (e.g., class 1 pharmaceuticals), pharmacist- or drug dealer-dispensed pharmaceuticals (e.g., class 2 and class 3 pharmaceuticals), which stored data are adjusted to practices in the country concerned. Optionally, data regarding whether medicines are in-stock (or out-of-stock) can be acquired by communicating with individual shops and the acquired data be stored in the shop database 253.

The display 26 is a device such as a liquid crystal display or a touch panel. The display 26 can display data on target route to a destination and proposals regarding health care such visiting a medical facility and purchasing medicine at a shop. The speaker 27 is a device for vocally outputting various information and can, for example, vocally announce target route information and the aforesaid health care information.

The ECU 10 is configured to include a computer having a CPU or other computing unit 120, a memory unit 110 consisting of ROM, RAM and/or similar, and other associated peripheral circuitry. The ECU 10 has a data storage unit 11 as a functional component of the memory unit 110, a route computing unit 12 as a functional component of the computing unit 120, a proposal determining unit 13, a retrieve unit 14, an output unit 15 and a response receiving unit 16.

The data storage unit 11 acquires passenger-specific biometric data measured by the measuring instruments 21 (measured data) and stores the measured data in association with measurement date and time. When the measuring instruments 21 are adapted to measure biometric data at predetermined intervals, the data storage unit 11 can be adapted to store only representative measured data rather than all of the measured data. Optionally, for example, measured data first measured after boarding or average value of multiple sets of measured data measured within a predetermined time period can be stored.

The route computing unit 12 uses current vehicle position measured by the GPS sensor 22 and map data stored in the map database 251 to compute a target route from current position to a destination. Control signals based on the results of the computation are output to the display 26 and the speaker 27 to display vehicle current position data and target route data on the display 26 and vocally output target route information from the speaker 27. Destination and other data related to route retrieval can be input through the input unit 24. Optionally, as discussed later, locations of medical facilities and shops proposed through the output unit 15 can, with passenger consent, be automatically set as destinations.

The proposal determining unit 13 determines which among multiple proposals regarding passenger health care, i.e., among different candidate proposals, should be offered to the passenger. The multiple proposals include advising a visit to the medical facility 2 or purchase of medicine at the shop 3 shown in FIG. 1. More specifically, the proposals regarding a visit to the medical facility 2 and purchase of medicine at the shop 3 include ones made on the outbound route RT1 on the way from departure point P1 toward destination P2 and ones made on the return route RT2 on the way back from the destination P2 to the departure point P1. In other words, the proposals include ones to receive an examination or purchase medicine by diverting from the outbound route RT1 along route RT1a or Rib of FIG. 1 and ones of receiving an examination or purchasing medicine by diverting from the return route RT2 along route RT2a or R2b. Proposals to purchase medicine include ones to buy a class 1 pharmaceutical, buy a class 2 pharmaceutical, and buy a class 3 pharmaceutical.

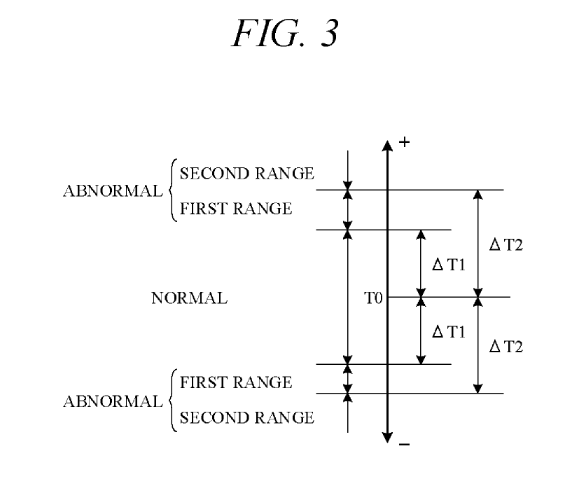

Based on biometric data measured and acquired by measuring instruments 21, the proposal determining unit 13 determines which among the aforesaid multiple proposals should be offered to the passenger. For example, when the biometric data acquired (measured) by the measuring instruments 21 is body temperature, passenger health condition is estimated by comparing the measured body temperature with a body temperature related threshold and the proposal to be offered is determined accordingly. Namely, the proposal determining unit 13 sets biometric data ranges (normal range, abnormal ranges) for determining proposals to be offered. A normal range is one in which health condition is determined to be good, and an abnormal range is one in which health condition is determined to be poor or deteriorating. FIG. 3 is a diagram showing examples of predefined normal and abnormal ranges. Such ranges are established for every biometric data category. The example in FIG. 3 is directed to the body temperature category.

As indicated in FIG. 3, body temperature within +ΔT1 from a predetermined center value (reference value) T0 (i.e., T0±ΔT1, e.g., 36° C.±1° C.) is defined as normal range, and body temperature outside this range as abnormal ranges. In other words, ΔT1 used as a threshold for determining whether passenger body temperature is normal, i.e., whether passenger health condition is good. The abnormal ranges are defined as first abnormal ranges falling outside the normal range but inside T0±ΔT2 (e.g., 36° C.±2° C.) and second abnormal ranges falling outside the first abnormal ranges. So ΔT2 is used as a threshold for determining degree to which passenger health condition is deteriorating, and passenger health condition is worse in the second abnormal ranges than in the first abnormal ranges.

For example, when measured body temperature is in a second abnormal range, the proposal determining unit 13 estimates that passenger health condition deterioration is severe. Since the passenger should immediately receive an examination at a medical facility in such a case, the proposal determining unit 13 determines to advise that the passenger visit a medical facility, e.g., that the passenger consult a doctor on the outbound route. On the other hand, when the measured body temperature is in a first abnormal range, the proposal determining unit 13 determines that the passenger health condition has not deteriorated to a degree requiring an immediate visit to a medical facility and, for example, determines to advise the passenger to consult a doctor on the return route or determines to propose that rather than visit a medical facility the passenger should buy medicine on the outbound or return route, preferably on the return route, i.e. when having more time freedom. When determining to propose that medicine be purchased, the proposal determining unit 13 also determines based on measured temperature which among class 1, class 2 and class 3 pharmaceuticals should be purchased. For example, it advises purchase of a class 1 pharmaceutical when measured body temperature is at or above a predetermined level and purchase of a class 2 pharmaceutical or class 3 pharmaceutical when it is below the predetermined temperature.

Taking into account that individual passengers have unique biometric characteristics and may, for example, have different (higher, for instance) normal and basal body temperatures, the normal, and first and second abnormal ranges are appropriately set using data stored in the data storage unit 11. For example, when five previous body temperatures measured and stored in the data storage unit 11 are all 36° C. and measured body temperature is 38° C., the proposal determining unit 13 sets reference value T0 to the average value 36.3° C. of the 6 measurements. When a measured body temperature is not in normal range (is in an abnormal range), average value calculated excluding the abnormal value can be set as reference value T0. For example, when 38° C. in the foregoing example is determined to be an abnormal value, the average value 36° C. of the preceding five measurements, i.e., excluding the abnormal value, can be set as reference value T0. Alternatively, instead of setting reference value T0 based on previous biometric data, ΔT1 and ΔT2 can be appropriately set based on previous biometric data. When the biometric data are in normal range, the proposal determining unit 13 determines no proposal regarding health care is necessary and does not determine a proposal to be made.

The retrieve unit 14 responds to the gist of a determination by the proposal determining unit 13 by retrieving a medical facility or shop to be proposed to the passenger concerned. For example, when a proposal advising a hospital visit on the outbound route is determined, the retrieve unit 14 accesses the medical facility database 252 and retrieves data of a medical facility convenient to visit on the outbound route. On the other hand, when a proposal advising purchase of medicine on the return route is determined, the retrieve unit 14 accesses the shop database 253 and retrieves data of a shop convenient to drop by on the return route.

The output unit 15 outputs control signals to the display 26 and the speaker 27 to inform the passenger of the proposal determined by the proposal determining unit 13. Optionally, the data retrieved by the retrieve unit 14 can be concurrently presented. When the output unit 15 proposes that a passenger visit a medical facility, an instruction asking the passenger to input acceptance or refusal of the proposal is displayed on the display 26 and output from the speaker 27. For example, the passenger is requested through the display 26 and speaker 27 to use the microphone 23 or the input unit 24 to input an instruction-in-response accepting or refusing the proposal.

The response receiving unit 16 receives the passenger's instruction-in-response (proposal acceptance or refusal instruction) in reply to the output from the output unit 15. Optionally, the output unit 15 can apply for a reservation at the medical facility when the response receiving unit 16 receives an instruction-in-response accepting the proposal to visit a medical facility. On the other hand, when the response receiving unit 16 receives a refusal of the proposal to visit a medical facility, the proposal determining unit 13 determines purchase of medicine at a shop as the proposal to be offered to the passenger, and the output unit 15 outputs control signals so as to output that proposal from the display 26 and the speaker 27. Optionally, when the response receiving unit 16 receives proposal acceptance, the proposal determining unit 13 can determine the value of the measured data from the measuring instruments 21 to be abnormal and establish normal range of the biometric data (such as reference value T0 of FIG. 3) based on measured data excluding the abnormal value. On the other hand, when the response receiving unit 16 receives proposal refusal, value of the measured data can be determined not to be abnormal, i.e., to be in normal range, and normal range can be established by including the measured data.

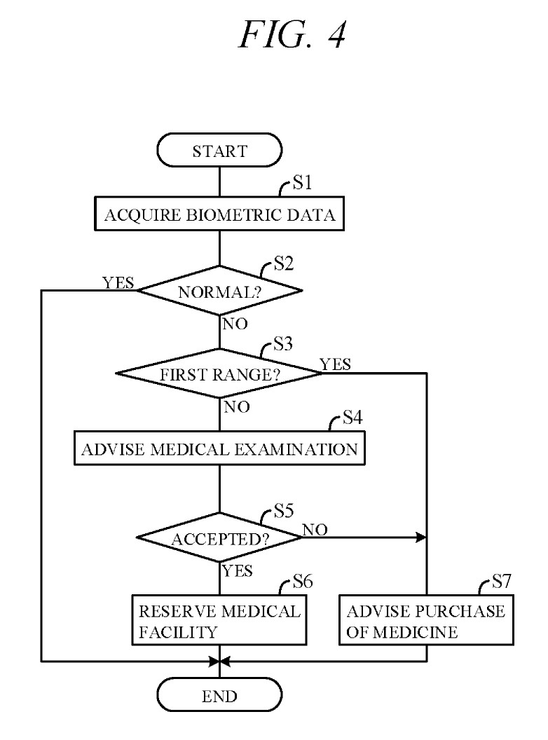

FIG. 4 is a flowchart showing an example of processing performed by the ECU 10 (CPU) in accordance with a program loaded in memory beforehand. The processing shown in this flowchart is started, for example, when the measuring instruments 21 perform health condition measurement following passenger vehicle boarding. First, in S1 (S: processing Step), current biometric data measured by the measuring instruments 21 and previous biometric data stored in the data storage unit 11 are acquired. Body temperature is taken as an example of biometric data in the following explanation.

Next, in S2, the proposal determining unit 13 determines whether current biometric data value is in a predefined normal range, i.e., whether health condition is good. When the result in S2 is NO, the program goes to S3, and when YES, processing is terminated. In S3, degree of health condition decline, i.e., whether biometric data value is in one of the first abnormal ranges, is determined. When the result in S3 is NO, the program goes to S4. Since biometric data value is in one of the second abnormal ranges in this case, passenger health condition deterioration is determined to be severe and a proposal advising examination at a medical facility is determined. In addition, the output unit 15 outputs control signals corresponding to the gist of the proposal to the display 26 and the speaker 27. As a result, the passenger can be advised by visual and audio output to visit a medical facility.

Next, in S5, the response receiving unit 16 determines whether the passenger accepted the proposal, i.e., whether a passenger instruction-in-response accepting the proposal was input through the microphone 23 or the input unit 24. When the result in S5 is YES, the program goes to S6, in which the output unit 15 asks a medical facility for a reservation, for example, and applies for a reservation at the medical facility. Concurrently, the route computing unit 12 computes a target route with the medical facility as the destination and outputs target route data through the display 26 and the speaker 27.

On the other hand, when the result in S3 is YES or the result in S5 is NO, the program goes to S7. In S7, the proposal determining unit 13 determines a proposal advising purchase of medicine at a shop. The proposal specifically advises purchase of one among a class 1 pharmaceutical, class 2 pharmaceutical and class 3 pharmaceutical. In addition, the retrieve unit 14 conducts a search to find a shop that handles the pharmaceutical concerned and acquires related shop information. Next, the output unit 15 informs the passenger through the display 26 and the speaker 27 of the proposal advising purchase of the pharmaceutical and the information regarding the shop that handles the pharmaceutical, namely, the shop's name, address, business hours and so on.

Concurrently, the route computing unit 12 computes a target route with the shop as the destination and outputs target route data through the display 26 and the speaker 27.

The present embodiment can achieve advantages and effects such as the following:

(1) The passenger health management apparatus 100 includes: the measuring instruments 21 for acquiring biometric data by measuring health condition of an in-vehicle passenger; the proposal determining unit 13 responsive to biometric data acquired by the measuring instruments 21 for determining which among multiple proposals regarding passenger health care, i.e., among multiple proposals including ones that advise a visit to a medical facility and ones that advise purchase of a pharmaceutical at a shop that sells pharmaceuticals, should be offered to the passenger; and the output unit 15, and display 26 and speaker 27 for informing the passenger of the proposal determined by the proposal determining unit 13 (FIGS. 1 and 2). With this configuration, since a passenger can be offered either a proposal advising a visit to a medical facility or a proposal advising purchase of a pharmaceutical at a store, the passenger can be given a proposal well matched to his or her health condition. In other words, proposals regarding health care that can be offered to a patient include both advising a visit to a medical facility and advising purchase of a pharmaceutical, so that a proposal optimally matched to health condition measured in a vehicle can be offered.

(2) The passenger health management apparatus 100 further includes the data storage unit 11 for storing biometric data acquired by the measuring instruments 21 (FIG. 2). The proposal determining unit 13 determines the proposal to be offered to the passenger based on previous biometric data stored in the data storage unit 11 and current biometric data acquired by the measuring instruments 21 (S4 and S7). A proposal can therefore be even more optimally offered to the passenger because the gist of the proposal to the passenger is determined with consideration to the passenger's unique biometric characteristics (e.g., normal body temperature, basal body temperature, and the like).

(3) The proposal determining unit 13 uses biometric data obtained by the measuring instruments 21 to estimate degree of passenger health condition decline and determines the proposal to be offered to the passenger in accordance with the estimated degree of health condition decline (S3, S4 and S7). Since this makes it possible, for example, to propose a visit to a medical facility when degree of health condition decline is severe and propose purchase of medicine when degree of decline is mild, the passenger can receive appropriate advice matched to degree of health condition decline.

(4) The multiple proposals determinable by the proposal determining unit 13 include ones advising medical examination or purchase of medicine on the outbound route to the destination and ones advising medical examination or purchase of medicine on the return route back from the destination. Therefore, when degree of health condition decline is mild (symptoms minor), for example, purchase of medicine on the return route (on way home, for example) rather than on the outbound route on the way to the destination (on way to work, for example) can be proposed. This means that in such a case the passenger can be advised to purchase medicine on the way home when less pressed for time and that the shop visit can be suitable timed.

(5) The multiple proposals determinable by the proposal determining unit 13 further include ones advising purchase of a class 1 pharmaceutical, class 2 pharmaceutical or class 3 pharmaceutical. When the proposal determining unit 13 determines purchase of a class 1 pharmaceutical, class 2 pharmaceutical or class 3 pharmaceutical as the proposal to be offered to the passenger, the output unit 15 outputs control signals to the display 26 and the speaker 27 so as to additionally provide the passenger with information regarding a shop that handles the determined pharmaceutical (S7). Purchase of a medicine suitable for the passenger's symptoms can therefore be proposed. Since the passenger can also be informed of a shop that handles the medicine, the passenger can efficiently visit the shop.

(6) The passenger health management apparatus 100 is equipped with the microphone 23 and the input unit 24 for inputting responses instructing acceptance or refusal of proposals presented through the display 26 and the speaker 27. When a visit to a medical facility was determined as the proposal to be offered to the passenger and that proposal is thereafter refused by the passenger, the proposal determining unit 13 then determines purchase of a pharmaceutical at a shop as the next proposal to be offered to the passenger (S5→S7). Therefore, when the passenger refuses a proposal to go to a medical facility (first proposal) for whatever reason, such as not having time for a hospital visit, disliking hospitals or thinking a visit too bothersome, the passenger can be offered a proposal to purchase medicine at a shop (second proposal), whereby the passenger can receive helpful health care information.

In the aforesaid embodiment, measurement of passenger health condition, determination of proposals to be offered to the passenger, and presentation of the proposals is performed by the passenger health management apparatus 100 installed in the vehicle, but, for example, determination of proposals to be offered to the passenger can be performed by a separate apparatus that can communicate with the onboard unit. In other words, it is optionally possible to build a client-server type passenger health care system and have the server perform some of the functions of the passenger health management apparatus 100 (FIG. 2).

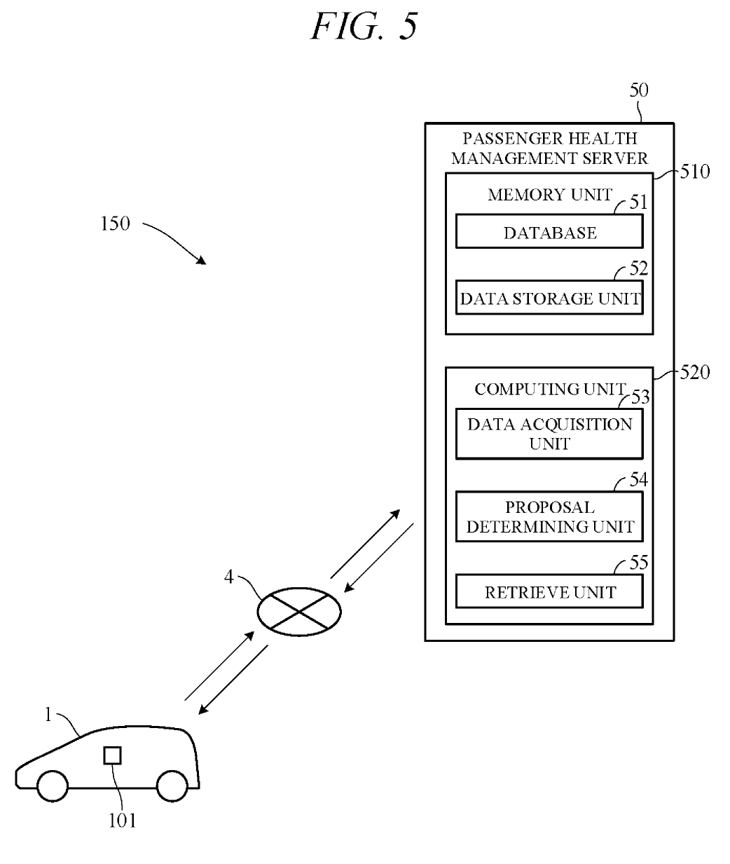

FIG. 5 is a block diagram showing an example of the overall configuration of a passenger health management system 150 in this case. As shown in FIG. 5, the passenger health management system 150 comprises an onboard unit 101 installed in the vehicle 1 and a passenger health management server 50. The onboard unit 101 and the passenger health management server 50 are communicatively connected through a network 4 including a wireless communications network. The onboard unit 101 is configured by omitting the database 25, and the data storage unit 11, proposal determining unit 13 and retrieve unit 14 of the ECU 10, from the passenger health management apparatus 100 of FIG. 2. Aside from these omissions, the onboard unit 101 is configured the same as the passenger health management apparatus 100.

The passenger health management server 50 is configured to include a computer having a CPU or other computing unit 520, a memory unit 510 consisting of ROM, RAM and/or similar, and other associated peripheral circuitry. The passenger health management server 50 comprises a database 51 and a data storage unit 52 as functional components of the memory unit 510, and comprises a data acquisition unit 53, a proposal determining unit 54 and a retrieve unit 55 as functional components of the computing unit 520. The data acquisition unit 53 communicates with the onboard unit 101 through an unshown wireless unit to acquire various data including biometric data measured and obtained by the measuring instruments 21. The database 51, data storage unit 52, proposal determining unit 54 and retrieve unit 55 are configured the same as the database 25, data storage unit 11, proposal determining unit 13 and retrieve unit 14 of the passenger health management apparatus 100 of FIG. 2.

The passenger health management server 50 is not limited to the aforesaid configuration insofar as it has a configuration corresponding to the proposal determining unit 54 by which it acquires biometric data measured and obtained by the measuring instruments 21, determines based on the acquired biometric data which among multiple proposals regarding passenger health care, i.e., among multiple proposals including ones that advise a visit to a medical facility and ones that advise purchase of a pharmaceutical at a shop that sells pharmaceuticals, should be offered to the passenger. Moreover, the onboard unit 101 is not limited to the aforesaid configuration insofar as it has a configuration corresponding to the measuring instruments 21 for acquiring biometric data by measuring health condition of a passenger in the vehicle and has a configuration corresponding to a presentation unit for acquiring the proposal determined by the proposal determining unit 54 and presenting the acquired proposal to the passenger, i.e., corresponding to the output unit 15, display 26 and speaker 27 of FIG. 2.

The aforesaid embodiments include not only the passenger health management apparatus 100 and the passenger health management system 150 but also the passenger health management method. The passenger health management method can be of any configuration insofar as it acquires biometric data by measuring health condition of an in-vehicle passenger, determines based on the acquired biometric data which among multiple proposals that are multiple proposals regarding passenger health care including ones that advise a visit to a medical facility and ones that advise purchase of a pharmaceutical at a shop that sells pharmaceuticals should be offered to the passenger, and presents the determined proposal to the passenger.

Although various proposals that can be determined by the proposal determining units 13 and 54 are cited in the forgoing, the proposals regarding health care to be offered to the passenger can include any of various proposals insofar as they include at least ones that advise a visit to a medical facility and ones that advise purchase of medicine at a shop that sells pharmaceuticals. In the aforesaid embodiments, proposals regarding health care are presented through the display 26 and the speaker 27 by means of control signals from the output unit 15, but the presentation unit is not limited to this configuration. In the aforesaid embodiments, a response input by a passenger for instructing acceptance or refusal of an offered proposal (an instruction-in-response) is input through the microphone 23 or the input unit 24, but the instruction-in-response unit is not limited to this configuration. In the aforesaid embodiments, the measuring instruments 21 provided in the vehicle measure body temperature, blood pressure and other biometric data, but biometric data to be measured is not limited to those stated in the foregoing, and the measuring unit is therefore not limited to the aforesaid configuration.

The above embodiment can be combined as desired with one or more of the above modifications. The modifications can also be combined with one another.

According to the present invention, since candidate proposals regarding health care that can be offered to a patient include both advising a visit to a medical facility and advising purchase of a pharmaceutical at a shop, the passenger can be offered a proposal optimally matched to the passenger's health condition measured in the vehicle.

Above, while the present invention has been described with reference to the preferred embodiments thereof, it will be understood, by those skilled in the art, that various changes and modifications may be made thereto without departing from the scope of the appended claims.

What is claimed is:

1. A passenger health management apparatus, comprising:
a measuring unit configured to measure health condition of a passenger onboard to acquire a biometric data;
a CPU and a memory coupled to the CPU; and
a presentation unit configured to preform presentation to the passenger,
the CPU and the memory being configured to determine a proposal to be presented to the passenger from among a plurality of proposals regarding passenger health management including a proposal advising medical examination at a medical facility and a proposal advising purchase of medicine at a shop that sells medicine based on the biometric data acquired by the measuring unit, wherein
the presentation unit is further configured to present the proposal to the passenger.

2. The passenger health management apparatus according to claim 1, wherein
the memory is further configured to store the biometric data acquired by the measuring unit, and
the CPU and the memory are configured to determine the proposal to be presented to the passenger based on the biometric data previously acquired by the measuring unit and stored in the memory and the biometric data currently acquired by the measuring unit.

3. The passenger health management apparatus according to claim 1, wherein
the CPU and the memory are configured to estimate a degree of passenger health condition decline based on the biometric data acquired by the measuring unit and to determine the proposal to be presented to the passenger in accordance with the estimated degree of health condition decline.

4. The passenger health management apparatus according to claim 3, wherein
the plurality of proposals further includes a proposal advising medical examination and purchase of medicine on an outbound route to a destination and a proposal advising medical examination and purchase of medicine on a return route back from the destination.

5. The passenger health management apparatus according to claim 3, wherein
the plurality of proposals further includes a proposal advising purchase of a first medicine which requires counter selling by a pharmacist and a second medicine which requires counter selling by a pharmacist or a dealer, and
the presentation unit is further configured to, when a proposal advising purchase of the first medicine or the second medicine is determined as the proposal to be presented to the passenger, present information on a shop dealing determined medicine along with the determined proposal to the passenger.

6. The passenger health management apparatus according to claim 1, wherein
the biometric data is a data on physical quantities indicating passenger health condition, and
the CPU and the memory are configured to set a normal range for the physical quantities in which passenger health condition is determined to be good, and to determine the proposal to be presented to the passenger based on a comparison result between the physical quantities currently acquired by the measuring unit and the normal range.

7. The passenger health management apparatus according to claim 6, wherein
the CPU and the memory are configured to set the normal range based on the physical quantities previously acquired by the measuring unit and stored in the memory.

8. The passenger health management apparatus according to claim 1, further comprising:
an instruction-in-response unit configured to input a response by the passenger for instructing acceptance or refusal of the proposal presented by the presentation unit, wherein
the CPU and the memory are configured to determine a proposal advising purchase of medicine at a shop as a next proposal to be presented to the passenger when a response instructing refusal is input by the instruction-in-response unit after determining a proposal advising examination at a medical facility as the proposal to be presented to the passenger.

9. The passenger health management apparatus according to claim 8, wherein
the CPU and the memory are configured to determine that the physical quantities currently acquired by the measuring unit is outside the normal range when a response instructing acceptance is input by the instruction-in-response unit and to determine that the physical quantities currently acquired by the measuring unit is inside the normal range when a response instructing refusal is input by the instruction-in-response unit.

10. The passenger health management apparatus according to claim 9, wherein
the CPU and the memory are configured to set the normal range based only on the physical quantities determined to be inside the normal range from among the physical quantities previously acquired by the measuring unit and stored in the memory.

11. A passenger health management system, comprising:
an onboard unit having a measuring unit configured to measure health condition of a passenger onboard to acquire a biometric data; and
a passenger health management server configured to be communicative with the onboard unit, wherein
the passenger health management server has:
a CPU and a memory coupled to the CPU,
the CPU and the memory being configured to perform acquiring a biometric data acquired by the measuring unit, and determining a proposal to be presented to a passenger from among a plurality of proposals regarding passenger health management including a proposal advising medical examination at a medical facility and a proposal advising purchase of medicine at a shop that sells medicine based on the biometric data acquired by the measuring unit, wherein
the onboard unit further has a presentation unit configured to acquire the proposal and to present the acquired proposal to the passenger.

12. A passenger health management method, comprising:
measuring health condition of a passenger onboard and acquiring a biometric data;
determining a proposal to be presented to a passenger from among a plurality of proposals regarding passenger health management including a proposal advising medical examination at a medical facility and a proposal advising purchase of medicine at a shop that sells medicine based on the biometric data; and
presenting the proposal to the passenger.
